(19) United States
(12) Patent Application Publication
(10) Pub. No.: US 2023/0129490 A1
(43) Pub. Date: Apr. 27, 2023

(54) SIMULATOR, ARTIFICIAL HEART VALVE, METHOD OF MAKING ARTIFICIAL HEART VALVE, ARM, AND PREDICTIVE METHOD

(71) Applicant: Wenbo Zhou, London (GB)

(72) Inventor: Wenbo Zhou, London (GB)

(21) Appl. No.: 17/969,307

(22) Filed: Oct. 19, 2022

(30) Foreign Application Priority Data
Oct. 23, 2021 (GB) 2115282.2

(51) Int. Cl. G09B 23/32; G09B 23/30

(52) U.S. Cl. CPC G09B 23/32; G09B 23/303

(57) ABSTRACT

A simulator comprising a chamber container; a flexible simulation chamber disposed inside the chamber container and simulating a chamber of a living body; at least one chamber arm disposed inside the chamber container, a distal end of the chamber arm being fixed relative to a point on the simulation chamber and a position of the distal end being controllable from a proximal end; and control means for controlling a simulation procedure to simulate movement of the chamber of the living body, the control means being adapted to change the position of the distal end during the simulation procedure.

### CROSS-REFERENCE TO RELATED APPLICATIONS

This application claims priority to Great Britain Application having serial number 2115282.2, filed on Oct. 23, 2021, which is incorporated herein by reference in its entirety for all purposes.

### FIELD OF THE INVENTION

The present invention relates to a device to simulate heart surgeries and minimally invasive heart disease therapies, including all traditional and new repairs and implantation therapies, involving education, training, research, novel therapy development, and clinical applications. The present invention also relates to the simulation of, and simulators and prosthetics for, other organs and cavities in the human or animal body.

### BACKGROUND OF THE INVENTION

For decades bio-engineers have tried to predict, compare and optimize heart therapy results. One effective way is to rehearse a therapy, including open surgery and minimally invasive therapy such as transcatheter repair and implantation, in an in vivo or in vitro environment. The in vitro therapy simulation is to practice or develop a therapy in a controllable mechanical system. Previous in vitro systems have included an isolated animal heart, and a 3D printed human heart model, while more recent development of this technology has brought hydrodynamic mechanical systems with fluid-structure interaction. Although the most advanced existing simulators have been considered close to human structure and function, due to the lack of comprehensive heart functions simulated, current in vitro aetiology studies are often sub-optimal and there has been no clinical application such as clinical prognosis with the existing heart simulators.

Embodiments of the present invention will now be described by way of example only, with reference to the accompanying drawings, in which:

Embodiments of the present invention will now be described by way of example only, with reference to the accompanying drawings, in which:

FIG. 1 is a schematic drawing of a pre-existing heart simulator including a ventricle simulation block 1, an atrium chamber block 2 and pumps 3. The ventricle simulation block 1 comprises a mock-up ventricle chamber 12 and the atrium chamber block 2 comprises a mock-up atrium chamber 22. The mock-up ventricle chamber 12 and the mock-up atrium chamber 22 are connected on one side by a simulation mitral valve and the other side by a compliance chamber 4. The pumps 3 control the external pressure applied to the mock-up ventricle chamber 12 and the mock-up atrium chamber 22 to simulate the beating of the heart. This illustrated simulator simulates either the left heart or the right heart (that is, the left-side or the right-side of the heart).

FIG. 2 is a schematic drawing of another pre-existing heart simulator which lacks a heart chamber beating function. Instead, in the line connecting the input of the mock-up atrium chamber and the output of the mock-up ventricle chamber there are provided the compliance chamber 4 and a mock-up blood pump 31 in series. Arms 112 extending outside the ventricle simulation block 1 are fixed in place at the beginning of the simulation to control the movement of the leaflets of the valve and hence simulate a fixed displacement of the papillary muscle (PM).

Heart valve regurgitation is the most frequent valvular heart disease. One main cause of heart valve regurgitation is abnormal papillary muscle (PM) displacement. Previous researchers have proven that in an in vitro simulator each one of PM position and ventricle geometry can make a difference to the simulation result. However, current heart simulators with mitral valve (MV) or tricuspid valve (TV) structure cannot simulate full heart valve function, due to the lack of either a PM position adjustment function or the left ventricle’s (LV’s) contrasting function of beating, the two being essential physiological heart functions which have hitherto been incompatible in heart simulators.

This limitation in prior art simulators is a result of incompatibility of the two functions in mechanical systems. The arm 112 has been made small to fit in the limited working space in the simulation blocks 1 and 2, and it is not possible to control a wide range of PM tip’s position inside the heart chamber without disturbing the LV’s physiological beating movement.

Although a few recent simulators have simulated LV chamber’s beating function, their simulated beating only mimics symmetrical deformation of a heart-shaped balloon.

In fact, a human’s left ventricle does not move symmetrically and such simulators do not mimic the physiological movement of the left ventricle and do not have the desired accuracy.

### SUMMARY OF THE INVENTION

The present invention seeks to address the shortcomings of prior art simulators and to provide both improved PM position control and LV physiological movement.

In the present invention, the previously conflicting functions of PM adjustment and LV beating are combined within one single simulator. This invention brings in vitro patient-specific prognosis one step closer to its clinical application, and makes future in vitro aetiology study’s simulation environment closer to a real human’s physiological condition.

In addition, in a simulator of the present invention, the LV rotates about a vertical (basal-apical) axis slightly periodically in synchronization with periodical beating movement.

According to a first aspect of the present invention, there is provided a simulator comprising: a chamber container; a flexible simulation chamber disposed inside the chamber container and simulating a chamber of a living body; at least one chamber arm disposed inside the chamber container, a distal end of the chamber arm being fixed relative to a point on the simulation chamber and a position of the distal end being controllable from a proximal end; and control means for controlling a simulation procedure to simulate movement of the chamber of the living body, the control means being adapted to change the position of the distal end during the simulation procedure.

Such a simulator allows accurate simulation of movement of a chamber of the living body, for example the left or right ventricle of the human heart in which case accurate rotation and/or other movement of the chamber during beating can be simulated.

According to second aspect of the present invention, there is provided a simulator comprising: a flexible simulation chamber simulating a chamber of a living body, the simulation chamber having at least one opening; a valve comprising leaflets; a simulation papillary muscle; and chordae connecting the simulation papillary muscle and the leaflets; at least one valve arm extending through the simulation chamber, so that the distal end of the valve arm is disposed in the simulation chamber and proximal end is disposed outside the simulation chamber, a distal end of the valve arm being fixed relative to the simulation papillary muscle and a position of the distal end of the valve arm being controllable from a proximal end; and control means for controlling a simulation procedure.

Such a simulator allows accurate control of the simulation papillary muscle for simulation, despite the use of a flexible chamber.

Preferably, the position of the distal end is adjustable to simulate end-systole positions of the papillary muscle of the living body.

Preferably, the control means is adapted to change the position of the distal end of the valve arm during the simulation procedure.

Preferably, the position of the distal end of the valve arm can be controlled without affecting movement of the simulation chamber.

Preferably, the valve arm comprises: a first rotational joint disposed inside the simulation chamber; first fixing means towards the distal end for fixing to the simulation papillary muscle; and a first extendable rod between the first rotational joint and the fixing means.

Preferably, the valve arm further comprises: a second rotational joint towards the proximal end and disposed outside the simulation chamber; second fixing means between the first and second rotational joints for fixing to the simulation chamber; and a second extendable rod between the second rotational joint and the second fixing means.

Preferably, the simulator further comprises: a second simulation chamber, wherein the first and second simulation chambers are connected via the opening with the valve in between them.

Preferably, the simulator further comprises: a chamber container in which the simulation chamber is disposed.

Preferably, the proximal end of the valve arm is fixed to a wall of the chamber container; a control motor is disposed adjacent the wall of the chamber container and connected to the proximal end; and the control means is arranged to control the control motor to change the position of the distal end of the valve arm.

Preferably, the simulator further comprises: at least one chamber arm disposed inside the chamber container, a distal end of the chamber arm being fixed relative to a point on the simulation chamber and a position of the distal end being controllable from a proximal end, the control means being adapted to change the position of the distal end of the chamber arm during the simulation procedure.

In the first and second aspects of the invention, it is preferred that the proximal end of the chamber arm is fixed to a wall of the chamber container; a respective control motor is disposed adjacent the wall of the chamber container and connected to the proximal end; and the control means is arranged to control the control motor to change the position of the distal end of the chamber arm.

Preferably, the chamber arm comprises: a chamber arm rotational joint towards the proximal end; chamber arm fixing means towards the distal end for fixing to the simulation chamber; and a chamber arm extendable rod between the chamber arm rotational joint and the chamber arm fixing means.

Preferably, the chamber arm fixing means comprises plates arranged on opposite sides of the simulation chamber for clamping the simulation chamber.

Preferably, the chamber arm fixing means is attached to the extendable rod with a ball joint.

Preferably, the position of the or each rotational joint is controlled by respective joint controlling wires and the extension of the or each extendable rod is controlled by a respective extension controlling wire.

Preferably, the simulation chamber simulates the left ventricle; and the control means is arranged to control the position of the distal end of the chamber arm to cause the left ventricle to rotate about a basal-apical axis in synchronisation with a periodical beating movement of the ventricle during the simulation procedure.

Preferably, the simulator is for simulating at least one of the left heart and the right heart.

According to another aspect of the invention, there is provided an artificial heart valve comprising: a ring; a leaflet attached to the ring; chordae; and an attachment block; and wherein ends of the chordae are attached to the leaflet and the attachment block respectively, the leaflet is formed of a first material, the chordae are formed of a second material, the attachment block is formed of a third material, and a covering layer is moulded around the first, second and third materials and is formed of a fourth material different from the first, second and third materials.

Preferably, the valve further comprises: a plurality of leaflets and a plurality of attachment blocks, wherein each of the attachment blocks is connected to each of the leaflets by the chordae.

Preferably, the first, second and third materials are different from each other.

Preferably, the first material is a cloth material.

Preferably, the second material is a braided wire.

Preferably, the third material is a solid plastic.

Preferably, the fourth material is silicone.

Preferably, the first, second and third materials are stitched together before the covering layer is moulded.

Preferably, the artificial heart valve is a mitral valve.

Preferably, the artificial heart valve Is a prosthesis for a human.

According to yet another aspect of the present invention, there is provided a method of making an artificial heart valve according to the previous aspect, the method comprising: forming a mould; positioning the first, second and third materials in the mould; and filling the mould with the fourth material to form the covering material around the first, second and third materials.

Preferably, the method further comprises: obtaining a scan of a corresponding valve of an individual, wherein the mould is formed and the first, second and third materials are positioned in the mould so that the positions of the connection points between the chordae and the attachment block and between the chordae and the leaflet match the those in the individual.

According to a yet further aspect of the present invention, there is provided a chamber arm for a simulator having a simulation chamber for simulating a chamber of a living body, the arm comprising: first fixing means towards the distal end for fixing to a portion of the simulation chamber; a first rotational joint towards the proximal end; and a first extendable rod between the first rotational joint and the first fixing means, wherein a position of the distal end is controllable from a proximal end.

Preferably, the chamber arm further comprises: wall fixing means for fixing the proximal end of the valve arm to a wall of a chamber container containing the simulation chamber.

Preferably, the position of the first rotational joint is controlled by joint controlling wires and the extension of the first extendable rod is controlled by an extension controlling wire.

Preferably, the chamber arm further comprises: a control motor at the proximal end, the control motor being controllable to change the position of the distal end of the arm.

Preferably, the fixing means comprises plates arranged on opposite sides of the simulation chamber for clamping the simulation chamber.

Preferably, the first fixing means is attached to the first extendable rod with a ball joint.

According to a yet further aspect of the present invention, there is provided a valve arm comprising: a chamber arm according to the previous aspect; a second extendable rod between the first fixing means and the distal end of the valve arm; second fixing means towards the distal end of the valve arm for fixing to a simulated papillary muscle in the simulation chamber; and a second rotational joint between the second extendable rod and the first fixing means.

Preferably, the position of the second rotational joint is controlled by respective joint controlling wires and the extension of the second extendable rod is controlled by a respective extension controlling wire.

According to another aspect of the present invention, there is provided a method for predicting the outcome of a proposed surgical treatment of an organ of an individual with a symptomatic condition, the method comprising: obtaining a scan of the organ; forming at least one first mock-up component based on the scan for simulating the organ in the symptomatic condition; running a first simulation using the first mock-up component and organ function parameters obtained from the patient in the symptomatic condition to obtain first illness severity indicators; running a second simulation by adjusting the first simulation until the first severity indicators match the medical records of the patient in the symptomatic condition; carrying out the proposed surgical treatment on the second simulation and obtaining second illness severity indicators; and determining the effectiveness of the proposed surgical treatment by comparing the second illness severity indicators to the medical records of the patient in a healthy or previous condition.

Preferably, the mthod further comprises: forming at least one corresponding second mock-up component based on the scan for simulating the organ in a healthy or previous condition; prior to running the first simulation, running an initial simulation using the first mock-up component and organ function parameters obtained from the patient in the healthy or previous condition to obtain initial illness severity indicators; comparing the initial illness severity indicators with the medical records of the patient in the healthy or previous condition; adjusting the initial simulation until the initial illness severity indicators match the medical records of the patient in the healthy or previous condition; and determining the feasibility of the method by comparing the initial illness severity indicators to the medical records of the patient in a healthy or previous condition.

Preferably, the organ is the heart, the first and second mock-up components simulate the left or right heart, or a part of the left or right heart, and the first and second simulations simulate movement of the at least one of the left or right heart.

Preferably, the determining comprises running the first simulation after carrying out the proposed surgical treatment to simulate rest and exercise and assessing damage to the organ.

Preferably, the method further comprises: making a plurality of first components; running respective first and second simulations for each first component; carrying out different surgical treatments on the respective second simulations; and determining which of the different surgical treatments is most effective based on which of the respective second illness severity indicators best matches the medical records of the patient in a healthy or previous condition.

### BRIEF DESCRIPTION OF THE DRAWINGS

Embodiments of the present invention will now be described by way of example only, with reference to the accompanying drawings, in which:

FIG. 1 is a schematic drawing of a pre-existing heart simulator which lacks PM control function.

FIG. 2 is a schematic drawing of a pre-existing heart simulator which lacks heart chamber beating function.

FIG. 3 is a schematic drawing of a heart simulator according to the present invention.

FIG. 4 shows a portion of the image in FIG. 3 including a ventricle simulation block 1 and an atrium simulation block 2.

FIG. 5 shows the ventricle simulation block 1 in FIG. 4 with particular focus on the mock-up MV 15.

FIG. 6 shows the bottom right corner of 1 Left Ventricle (LV) Simulation Block [00163] in FIG. 4 with particular focus on the mock-up MV 15, highlighting the structural difference between brachium 13A, brachium 13B and forearm 14.

FIG. 7 is a 3D drawing of the brachium 13A focusing on the way it is connected with a wall of heart chamber container 11 or 21 and motor set 110.

FIG. 8 is a schematic view illustrating the components of the brachium 13A.

FIG. 9 is a detailed view of the clamping twin plates assembly 134 in FIG. 8.

FIG. 10 is a cross-section view of upper half of brachium 13A, focusing on the control mechanism and omitting the motor set 110 for ease of explanation.

FIG. 11 is to demonstrate brachium 13C with an optional ball joint 139 to connect the clamping twin plates assembly 134 and the rod 133C.

FIG. 12 is a detailed cross-section view of the ball joint 139 in FIG. 11.

FIG. 13 is a 3D drawing of the PM holder 111 which comprises brachium 13B and forearm 14.

FIG. 14 is a schematic view illustrating the components of the forearm 14.

FIG. 15 is a cross-section view of upper half of forearm 14, focusing on the control mechanism.

FIG. 16 has the same content as FIG. 6, focusing on the way how a PM 151 is attached to a PM holder 111.

FIG. 17 is a cross-section view of upper half of brachium 13B, focusing on the control mechanism.

FIG. 18 shows the simulation procedure of prognosis using the invention.

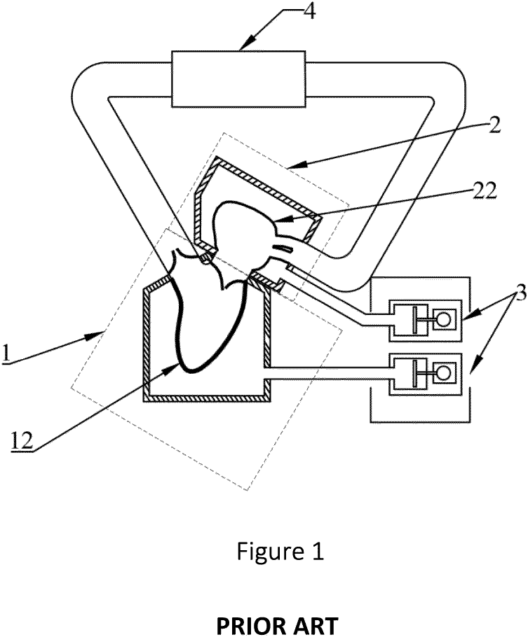

### DETAILED DESCRIPTION OF THE INVENTION

### Overview

The present invention provides apparatus and a method that allow an operator, who may be but not limited to a surgeon, a trainee, a researcher, a novel therapy developer and any medical professional, to simulate a therapy which could be either open surgery or minimally invasive therapy performed on a human organ with chamber structure, using organ simulator containing real-size mock-up organ. The organ simulator is capable to simulate patient-specific organ properties including geometry and the way of movement, at healthy, unhealthy, pre-operation and post-operation conditions.

A full therapy simulation procedure comprises a number of steps in time order:
To make mock-up organ chambers with patient-specific geometry obtained from clinical scans.
To simulate patient-specific healthy condition.
To simulate patient-specific symptom (unhealthy condition).
To carry out simulated therapy on the mock-up organ with symptom.
To simulate post-operation condition of the unhealthy mock-up organ.
If the post-operation condition is not satisfying, repeat all steps above with another or improved therapy, until an optimized result is observed.

Other than clinical application, the invention can also be used for development of novel therapies, aetiology research, education, training, and animal disease treatment. While used for non-clinical applications, the simulated organ could be in an animal’s geometry for animal study, or a generic geometry modified by CAD rather than a specific patient’s geometry, to better reflect a common geometry among a certain population.

The mock-up organ is made from tissue equivalent material (TEM), for example, a type of flexible and transparent silicone material such as Transil 40-1, on which the simulated therapies is to be performed.

Such mock-up organ can typically be a mock-up heart, while the simulator may be used, mutatis mutandis, for simulating therapies in other chamber-like body organs such as a lung. In this document, by way of example, mock-up organ is assumed to be a heart but the present invention is not limited thereto.

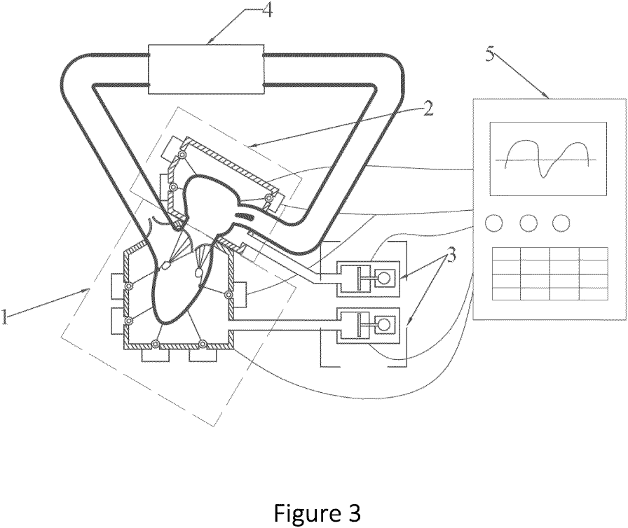

The organ simulator apparatus comprises the following functional assemblies as displayed in FIG. 3: the ventricle simulation block 1 simulating a beating ventricle chamber, the atrium simulation block 2 simulating a beating atrium chamber, a set of pumps 3 driving the beating of heart chambers, compliance system 4 simulating other parts of human circulatory system, and data system 5 which comprises a motor driving system (MDS) controlling all the motors in the simulator and a data acquisition system (DES) which monitors, processes, and reports simulator function data such as heartrate, blood pressure, cardiac output, regurgitant fraction, effective orifice area (EOA), and other heart performance indicators. Pressure sensors and flowmeters are also part of the data system 5.

The simulation blocks 1 and 2 are designed as part of this invention, while all other functional components of the simulator may typically be obtained from a hydrodynamic simulation platform such as ViVitro Pulse Duplicator System provided by ViVitro Labs, Inc. located at 455 Boleskine Road, Victoria, BC, Canada. The present invention, blocks 1 and 2 is fully compatible with the ViVitro Pulse Duplicator System.

This embodiment of the invention comprises two simulated heart chambers when there are four chambers in a human’s heart. The simulator is to simulate either the left heart or right heart. In either side of the heart, there is an atrium chamber on top of a ventricle chamber, with one heart valve inside the connection orifice between the two chambers to allow single-direction blood flow from the atrium to the ventricle, and the other heart valve in the blood outlet orifice on the ventricle. In this embodiment, by way of example, the left heart is assumed to be simulated, therefore, (listed in the order of blood flow) pulmonary vein (PV), left atrium (LA), mitral valve (MV), left ventricle (LV), aortic valve (AV) and aorta are simulated. Alternatively, when used for right heart simulation, each left heart simulator component can also simulate relevant components in the right heart, including superior vena cava (SVC), right atrium (RA), Tricuspid valve (TV), right ventricle (RV), pulmonic valve, and pulmonary artery (PA). Furthermore, the invention may also be used, mutatis mutandis, for simulating other chamber-like body organs such as lungs.

### Detailed Description

The chamber simulation blocks [00163]&2

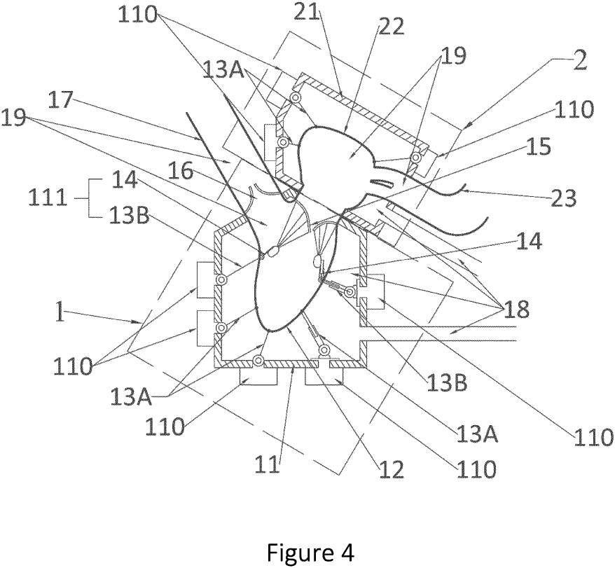

Reference is now made to FIG. 4, which shows a portion of the image in FIG. 3 including a ventricle simulation block 1 and an atrium simulation block 2.

The 1 Left Ventricle (LV) Simulation Block [00163] allows a human operator to perform a simulation of LV under various conditions, including pre- and post- therapy conditions with different health statuses.

The 1 Left Ventricle (LV) Simulation Block [00163]’s shell is LV chamber container 11, which is typically made of transparent plastic glass such as acrylic to allow clear observation and Particle Image Velocimetry (PIV) studies to the components inside. To simulate open chest surgery such as PM approximation associated with undersizing restrictive annuloplasty, PM relocation and LV remodelling, each board of the LV chamber container 11 can be disassembled as required by the operator to allow convenient space for operation. To simulate minimally invasive therapies such as transapical beating-heart chordae implantation in mitral regurgitation, a few boards of the LV chamber container 11 may be replaced with ones with specifically designed holes with rubber sealing to allow insertion of a medical device. A few openings may remain in the LV chamber container 11 for movement control assembly and pumps 3, which will be described in detail in contents below.

The mock-up LV chamber 12 simulates the movement of real LV. The mock-up LV chamber 12 is made in transparent and flexible material with similar mechanical properties to local tissue, such as silicone and rubber. The geometry and property of the mock-up LV chamber 12 can be modified to meet specific requirements of each simulation. For example, geometry generated with CAD as an average generic geometry of a specific population could be applied to study the aetiology of this population while a patient-specific geometry could be applied for prognosis, and a few rigid plastic pieces could be embedded into the wall of the mock-up LV chamber 12 to simulate calcification of local tissue.

The mock-up LV chamber 12 is manufactured with liquid material casting, with either a male mould alone with LV inner wall geometry, or a multi-part mould including male and female components when the female wall comes with LV outer surface geometry. When thickness control is needed to adjust area-specific mechanical properties, both male and female moulds are necessary. The moulds could be made by a milling machine or 3D printer. When a patient-specific geometry is required, clinical scans such as MRI needs to be carried out to generate the geometry file with a format compatible with the mould manufacturing machine, such as the “.stl” format, otherwise, CAD could be applied to modify the geometry file and thus adjust the simulated LV chamber.

The LA chamber container 21 has a similar or identical structure, function and material to the LV chamber container 11, with the only difference (or the main difference) between these two containers being the heart chamber (LV or LA) the container is containing. The mock-up LA 22 has a similar or identical manufacturing process, function and material to the mock-up LV chamber 12, except for that these two mock-up chambers simulate different chambers of the heart.

Arms 13A each control the dynamic position of a point on the surface of the mock-up LV chamber 12 and the mock-up LA chamber 22, during each pumping cycle phase, including diastole and systole. In the present description, each arm 13A is called a ‘brachium’ but it will be apparent that other types of rods, cylinders, arms or legs, whether or not jointed, may be used as arms 13A.

In addition, papillary muscle (PM) holders 111 control the dynamic position of the PMs 151. PM holders 111 each comprise a brachium 13B and a forearm 14. Again, the present invention is not limited to implementation using a brachium and forearm arrangement and it will be apparent to the skilled addressee that other arrangements of rods, cylinders, arms and legs, preferably jointed, may be used for the PM holders 111.

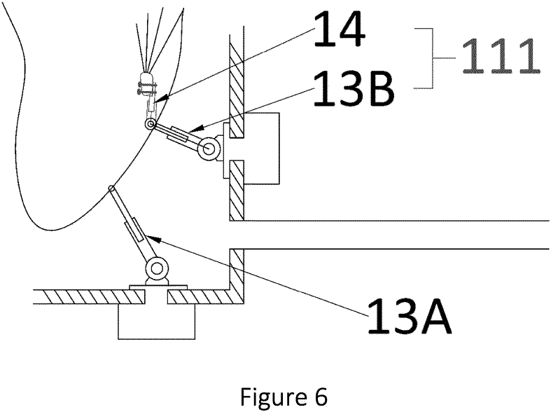

Reference is now made to FIG. 6, which shows the bottom right corner of 1

Left Ventricle (LV) Simulation Block [00163] in FIG. 4 with particular focus on the mock-up MV 15, highlighting the structural difference between brachium 13A, brachium 13B and forearm 14.

The brachium 13A’s motion is controlled by a motor set 110 mounted on the exterior surface of a heart chamber container 11 or 21 (see FIG. 4), through a mechanical driving system inside the brachium 13A. In a heart chamber container 11 or 21, at least five brachia 13A are applied to simulate the physiological movement of the heart chamber, including the dynamic rotation of the LV about the basal-apical axis.

The brachium 13B is a special type of brachium. In addition to all structures and functions of a brachium 13A, a brachium 13B is also capable of controlling a forearm 14 which is connected to the upper end of brachium 13B. More mechanical driving systems are inside brachium 13B in comparison with brachium 13A, enabling the motion control of forearm 14 by a motor set 110. A brachium 13B and a forearm 14 together form a PM holder 111, which is to control motions of the PMs 151, especially the dynamic position of PM tips as discussed below, and therefore control the severity of the simulated PM displacement disease. In mechanical control point of view, the PM tips inside the LV chamber container 11 are controlled independently, without affecting the movement of LV chamber container 11, and this is a novel function invention.

Although only two are illustrated, there are preferably three PM holders 111 in a multipurpose ventricle simulation block, which can be used as either a 1 Left Ventricle (LV) Simulation Block [00163] or a right ventricle (RV) simulation block. In this case, two of the three PM holders 111 may be used for simulation of the left heart where two PMs are in the LV, and all three PM holders 111 may be used for simulation of the right heart where three PMs are in the RV. Alternatively, specifically tailored simulation blocks with the appropriate number and position of PM holders 111 may be provided for the left and right ventricles respectively.

Returning to the description of this embodiment, LA Simulation Block 2 includes no PM holder 111.

The mock-up MV 15, AV 16 aorta 17 are typical structures of pre-existing simulators. In a simulator according to the present invention, these three components 15, 16 and 17 could either be pre-existing non-patient-specific products or fixed isolated animal tissue, or alternatively manufactured using the same material and method as the mock-up heart chambers 12 and 22. The casting process of mock-up MV 15 may alternatively be an upgraded process, which is discussed below and, is a unique and novel feature of the present invention.

The pumps 3 are used to drive the beating of the heart chambers by periodically pumping the media liquid 18 which is filled into the space between the silicone heart chambers 12 and 22 and their containers 11 and 21, rather than to pump the mock-up blood 19 inside the mock-up heart chambers 12 and 22 directly.

The mock-up MV 15

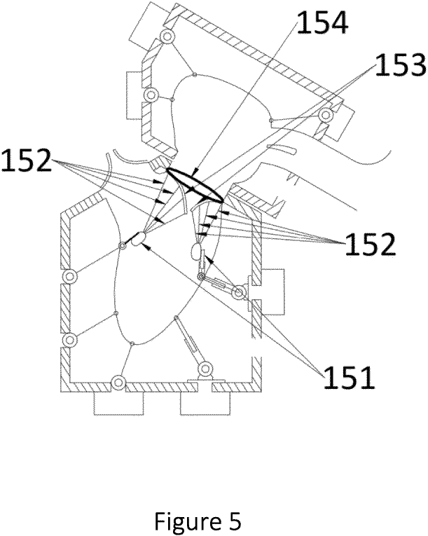

Reference is now made to FIG. 5 which shows the ventricle simulation block 1 in FIG. 4 with particular focus on the mock-up MV 15. The mock-up MV 15 is mounted between the orifice connecting the mock-up heart chambers 12 and 22, and comprises four components: papillary muscles (PM) 151, chordae 152, leaflets 153 and mitral annulus (MA) 154. Each PM 151 is controlled by a PM holder 111. The MA 154 is mounted between the orifice connecting the mock-up heart chambers 12 and 22. The leaflets 153 are held by the MA, and the chordae 152 connect the leaflets 153 and the PMs 151. More specifically, the chordae 152 are connected to the PMs 151 at the respective points on the surface of PM tips. Although not shown, each PM 151 is connected to both leaflets by the chordae 152. The chordae 152 and leaflets 153 in between the PM 151 and MA 154 are otherwise not attached to anything unless a therapy involves any change in these two parts 152 and 153, such as chordae and leaflet repair therapies.

The PM 151 in this embodiment is a solid block and does not itself contract and relax. Instead, it simulates the tip of the papillary muscle in that it is the attachment site for the chordae. Simulation of movement of the papillary muscle tip in this embodiment is carried out by moving the PM 151 using the PM holder 111. Consequently, the PM 151 may be considered an attachment block for attaching the chordae.

The manufacturing process of a mock-up MV 15 could be upgraded with an extra process, as follows. Before being cast with silicone inside a closed male and female mould system, some stronger additional material could be placed inside the mould where silicone is to be filled up. The additional material varies for different parts of the valve: cotton cloth tailored with desired shape for the leaflet, fishing wire for chordae, and 3D printed plastic solid with patient-specific PM tip geometry including the position of chordae-PM connection points. Before silicone casting, these placed additional materials must be connected to each other the same way as physiological connection, typically by stitching. After silicone casting, these additional materials will be embedded in a silicone layer. The 3D printed mould should preferably include details of the MV geometry such as the leaflet-chordae and chordae-PM connection points. A vacuum chamber may be employed to avoid the existence of bubbles during the casting process. The fishing wire is typically made of braided (Multifilament) spectra fibre and with the property of: no reel memory, abrasion resistance, at least 3N breaking stretch load. All other material must be strong enough so that the mock-up MV 15 can bear at least a 3N load.

The manufacturing methods and materials for the mock-up MV 15 applies to mock-up tricuspid valve as well, when the invention is used for right heart simulation.

Part breakdown brachium 13A

Reference is now made to the following three drawings:

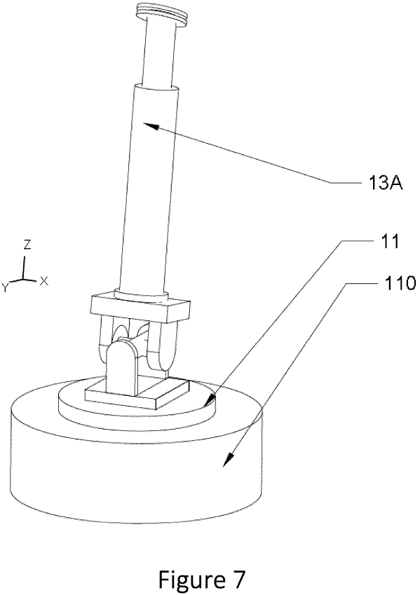

FIG. 7 which is a 3D drawing of the brachium 13A focusing on the way it is connected with a wall of heart chamber container 11 or 21 and motor set 110.

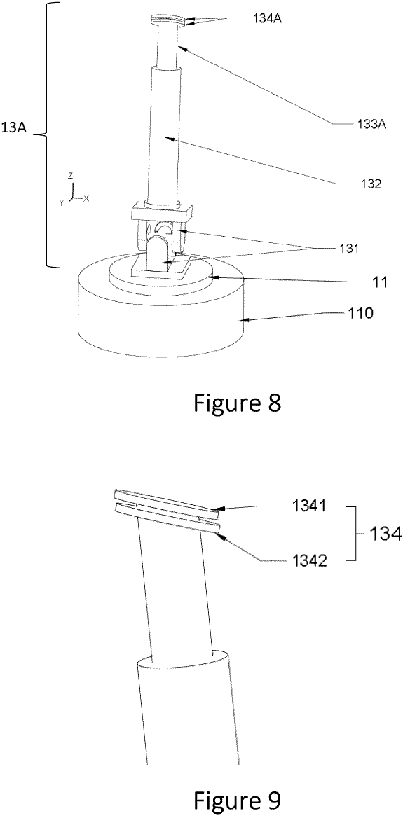

FIG. 8 which is a schematic view illustrating the components of the brachium 13A.

FIG. 9 which is a detailed view of the clamping twin plates assembly 134 in FIG. 8.

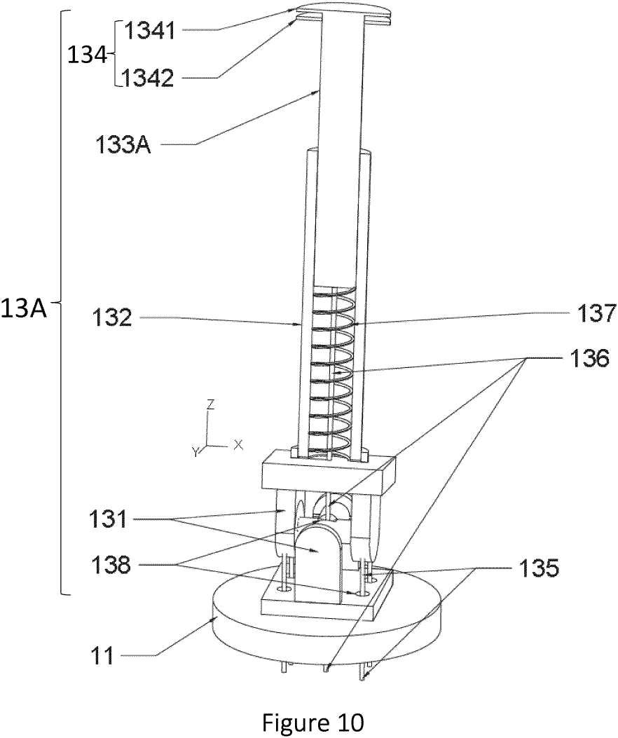

FIG. 10 which is a cross-section view of upper half of brachium 13A, focusing on the control mechanism and omitting the motor set 110 for ease of explanation.

In the following description, the attachment and use of the brachium 13A to and in the LV chamber container 11 will be described. However, it will be apparent that the brachium 13A can be attached to and used in the LA chamber container 21 in a similar way to control the LA mock-up chamber 22.

A brachium 13A and a motor set 110 are mounted on opposite sides of a wall of the chamber container 11 or 21 (FIG. 7). However, it will be apparent that part of the motor set 110 may be disposed inside chamber container 11 and/or part of the brachium 13A may extend outside chamber container 11. The brachium comprises a rotational joint 131 allowing 2D rotational control of the brachium 13A, including rotation about X and Y axis (see coordinate system at the left side of FIG. 8).

In an alternative embodiment, rotational joint 131 may be upgraded to have one more degrees-of-freedom rotation control rotate about the Z axis (not shown). Such upgrade could be, by way of example, a digital controlled ball joint with controls of rotation about all x, y and z axes.

The rod 133A is inserted in tube 132 forming an extendable brachium. The clamping twin plates assembly 134 is to clamp the wall of mock-up heart chambers 12 and 22 (FIG. 9) to achieve control of one point of the heart chamber surface. The clamping twin plates assembly 134 comprises a upper plate 1341 which is integrated as one piece with rod 133A and a lower plate 1342 which is an individual part. These two plates 1341 and 1342 are tightened with at least three threaded screws. The angle of clamping twin plates assembly 134 could be customized during manufacture process, to fit specific heart chamber 12 and 22 geometry.

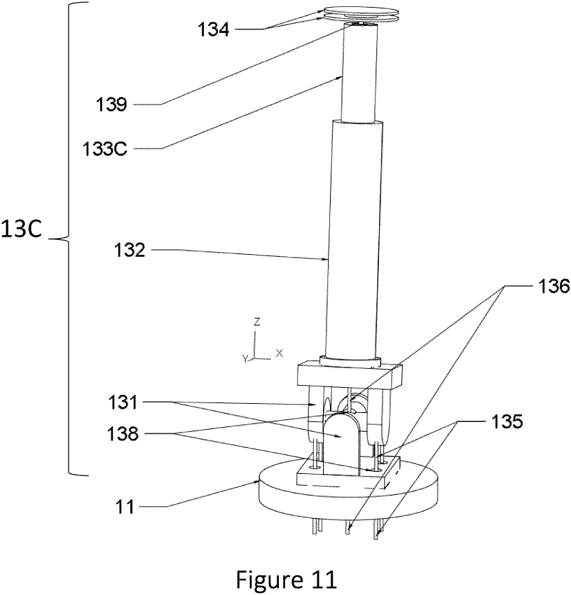

The rotational and extension degrees of freedom are controlled through a series of control mechanisms. As shown in FIG. 10, the rotational joint in the present embodiment includes two pivot j oints 131, allowing rotation about the X and Y axes respectively. As shown in FIGS. 10 and 11, but omitted from other figures for ease of explanation, four joint controlling wires 135 are attached to part holding the tube 132 to adjust rotational gesture about X and Y axes. In the figure, pulling on the front two wires 135 will cause the tube to rotate forwards about the X axis, pulling on the rear two wires 135 will cause the tube to rotate backwards about the X axis, pulling on the right two wires 135 will cause the tube to rotate to the right about the Y axis, and pulling on the left two wires 135 will cause the tube to rotate to the left about the Y axis. One extension controlling wire 136 is to control the pulling distance to the rod 133A to the Z- direction, against the pushing force provided by spring 137 to push the rod 133A to the Z+ direction.

The four joint controlling wires 135 and the extension controlling wire 136 can be pulled/released to adjusted lengths to cause the rod 133A and the clamping twin plates assembly 134 to move in any direction.

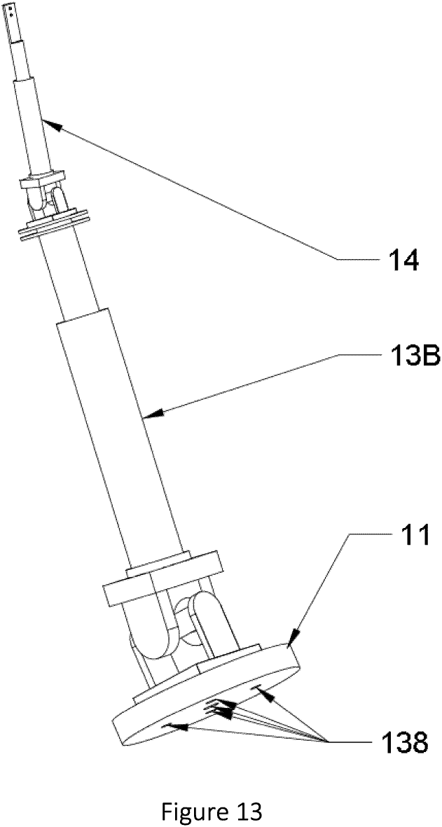

The wires 135 and 136 are omitted from FIG. 7, FIG. 8 and FIG. 13, and the motor set is omitted from FIG. 10 FIG. 11, for ease of explanation.

The joint controlling wires 135 and the extension controlling wire 136 extend through holes 138 in the joint 131 and the chamber container 11 or 21 to the motor set 110, which is operated to control the position of the clamping twin plates assembly 134. The holes 138 are designed to allow barrier-free sliding movement of all the wires 135 and 136, and the length of each one of these wires is controlled by a motor in the motor set 110.

The extension controlling wire 136 and spring 137 assembly could be replaced by any other extension control mechanism that can be digitally controlled, such as wheel gear system and hydraulic pump.

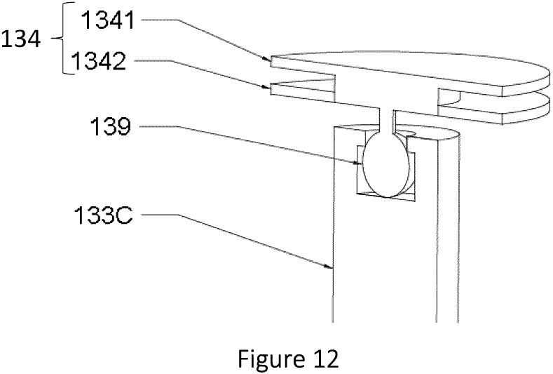

In a preferred brachium 13C shown in FIG. 11 and FIG. 12, ball joint 139 (FIG. 12) can be added between rod 133A and clamping twin plates assembly 134 to form clamping twin plates assembly 134. The ball joint has uncontrolled 3D degree-of-freedom, to release unnecessary strain caused to the controlled surface point of the mock-up heart chambers 12 and 22. Although not shown, the ball joint could be locked by inserting a threaded screw through the surface of rod 133A and pressing the surface of the ball in the ball joint 139, whenever required in a more precise simulation.

As with brachium 13A, the plates of the clamping twin plates assembly 134 are separable and three (or any suitable number) of screws (not shown) are used to clamp the plates together, holding the mock-up heart chamber 12, 22 between them. In other words, once fitted plate 1341 is inside the heart chamber 12, 22 and plate 1342 is outside it. Same arrangement applies to 13B and 13C.

Part breakdown of PM holder 111

Reference is now made to the following three drawings:

FIG. 13 which is a 3D drawing of the PM holder 111 which comprises brachium 13B and forearm 14.

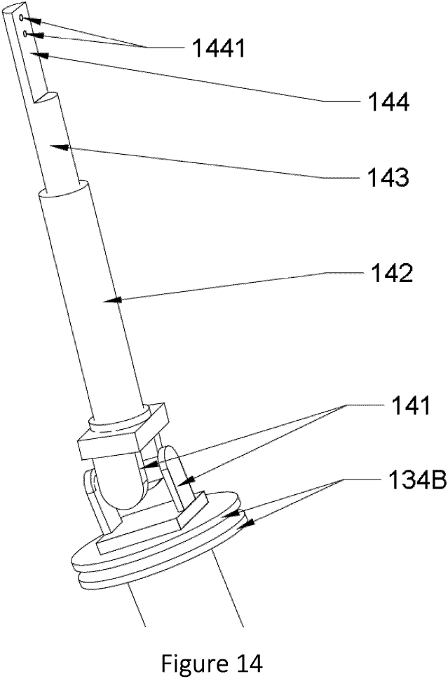

FIG. 14 which is a schematic view illustrating the components of the forearm 14.

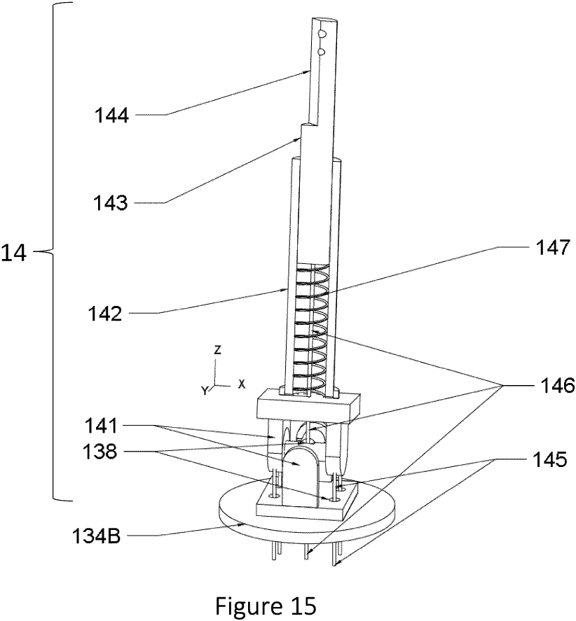

FIG. 15 is a cross-section view of upper half of forearm 14, focusing on the control mechanism.

A PM holder 111 comprises a brachium 13B and a forearm 14 (FIG. 13). The brachium 13B includes the same structure as the brachium 13A, but with additional fitting and control of the forearm 14. As shown in FIG. 4 and FIG. 6, the brachium 13B is attached to the LV chamber container 11 and is disposed between the chamber container 11 and the mock-up LV chamber 12. The upper and lower plates 1341, 1342 clamp the mock-up LV chamber 12. The forearm 14 is mounted on the surface of the upper plate 1341 of the clamping twin plates assembly 134 of the brachium 13B, and is located inside a mock-up heart chamber 12. The forearm 14 has similar structure (FIG. 14) and the same control mechanism (FIG. 15) as the brachium 13A.

The table below shows the same structure between the forearm 14 and the brachium 13A.

| | |
|---|---|
| In brachium 13A (FIG. 8 and FIG. 10 ) | In forearm 14 (FIG. 14 and FIG. 15 ) |
| rotational joint 131 | rotational joint 141 |
| tube 132 | tube 142 |
| rod 133A | rod 143 |
| joint controlling wires 135 | joint controlling wires 145 |
| extension controlling wire 136 | extension controlling wire 146 |
| spring 137 | spring 147 |

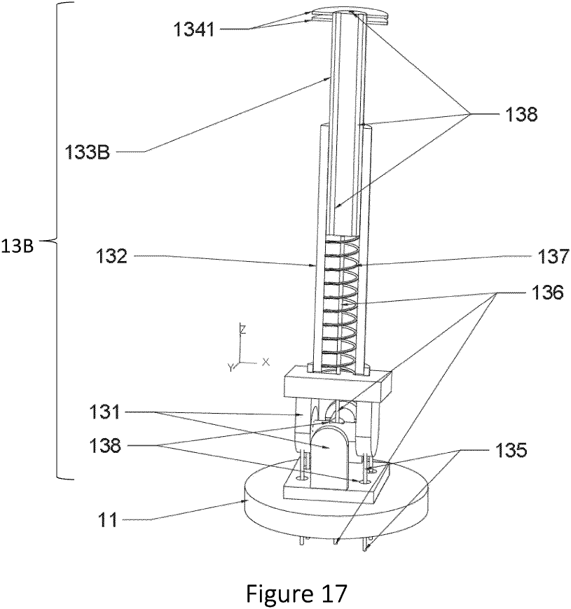

The joint controlling wires 145 and the extension controlling wire 146 extend through the clamping twin plates assembly 134, the rod 133A, the tube 132, the rotational joint 131, and the holes 138 in the joint 131 and wall of the LV chamber container 11 into the motor set 110, for control in a similar way to the brachium 13A. The wires 145, 146 are omitted from FIG. 13, FIG. 14, and FIG. 17, the brachium is omitted from FIG. 15, and the motor set is omitted from FIG. 13 FIG. 15 and FIG. 17, for ease of explanation.

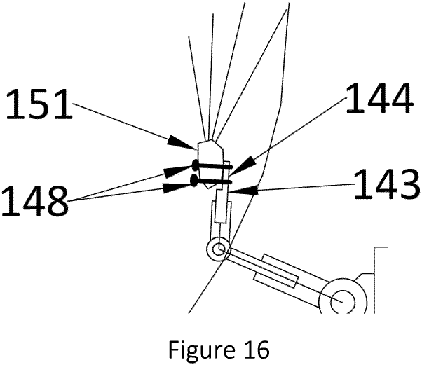

The only other main difference between the forearm 14 and the brachium 13A is the part above the rod 143. A connection pad 144 is glued on top of, or otherwise attached to, or integrally formed with rod 143. The connection pad 144 has at least two threaded holes 1441 so that PM 151 can be connected with the connection pad 144 firmly with screw 148 (FIG. 16).

The brachium 13B looks identical to the brachium 13A from outside (FIG. 13), because the only difference is the internal structure (FIG. 17). The brachium 13B has all components of the brachium 13A, and in addition, the brachium 13B also have the following components. The holes 138 drilled through the rod 133B is to allow the wires 145 and 146 controlling the gesture of forearm 14 to reach the motor set 110 and controlled by motors. The wires 145 and 146 also shares the route occupied by wires 135 and 136.

In an alternative way to control the gestures of brachium 13A 13B 13C and forearm 14, manual control of the wire lengths could replace the motor digital control, to effectively save costs, although dynamic position control of relevant parts is sacrificed. One typical application of this simplified apparatus is functional mitral regurgitation simulation caused by PM displacement, because the only moment when PM position has an impact on MV leaflet closure is at end of systole. In this application, keeping PM at a fixed position has been considered acceptable in academic research to date.

### Control for Dynamic Simulation

One function of the control system 5 is to apply control signal to drive the motor set 110 and provide precise control of dynamic position of LV LA surface and PMs.

Each motor controls a wire 135, 136, 145, or 146, and all motors together control the pulling distance of the wires which further controls the rotational degrees of joints 131 and 141, and the extension length of rods 133A, 133B, 133C and 143. The controlled joint and extension rods together realise the 3D position control of the free end of the brachium 13A 13B 13C and forearm 14.

The multi-degree-of-freedom movement of the brachia 13A 13B 13C enables multiple points on the chamber surfaces to move as controlled. The controlled points, together with the pumps 3 which control the pumping volume amplitude and frequency of heart chambers, drive the heart chamber to beat following physiological patient-specific movement. An example of periodical physiological movement of heart chamber, is the slight rotational movement of LV about the vertical (basal-apical) axis.

The driving signal must be periodically changing with reasonable precision to realise desired physiological heart chamber movement, similarly, patient-specific PM control is realised with the dynamic control of the forearm 14 which is attached by the mock-up PMs.

Importantly, from a mechanical control point of view, the PM tips inside the LV chamber container 11 can be controlled independently, without affecting the movement of LV chamber container 11, and this is a novel function invention.

A 3D scan such as MRI is to be applied to the heart simulator and a patient to compare the heart chamber movement and PM movement. Digital signal is adjusted accordingly to make sure the accuracy of the simulated movements. An MRI scan may be run for a predetermined period of time, for example a few seconds, to generate a 4D video, alternatively called a 4D scan. Either a 3D or 4D scan may be used in the invention.

As such, the control means 5 is able to control the ends of the brachia 13A, 13C (and hence the points of the chambers 12, 22 to which they are attached) to move periodically in three dimensions in synchronism with the beating of the heart during a simulation operation. They can also be moved to a predetermined position before the start of the simulation. In this way, for example, the LV can rotate about a vertical (basal-apical) axis slightly periodically in synchronization with periodical beating movement.

There may be two mock-up heart chambers and two affiliated valves with subvalvular structures, simulating patient’s heart structure of either the left heart or the right heart.

Preferably, the simulator has a patient-specific geometrical structure, functions, and malfunctions obtained from body scans, clinical tests and/or CAD, wherein abnormal tissue deformations causing functional disease are controlled by mechanical assemblies driven either digitally or manually, and abnormal tissue degeneration and calcification causing organic diseases are mimicked by tissue-equivalent material with patient-specific mechanical properties such as stiffness and strength and geometries including damaged geometry and other organic hear diseases.

A series of programmable motor groups is configured to apply dynamic patient-specific chamber tissue movements and deformations to the heart chambers by controlling the movement of mechanical assemblies, so as to simulate patient-specific heart performances during healthy conditions and functional heart disease including abnormal heart chamber dilation and PM displacement.

The present invention can be used to simulate healthy, unhealthy, pre-operational and post-operational conditions of specific patients and therapies.

### Simulation Procedure

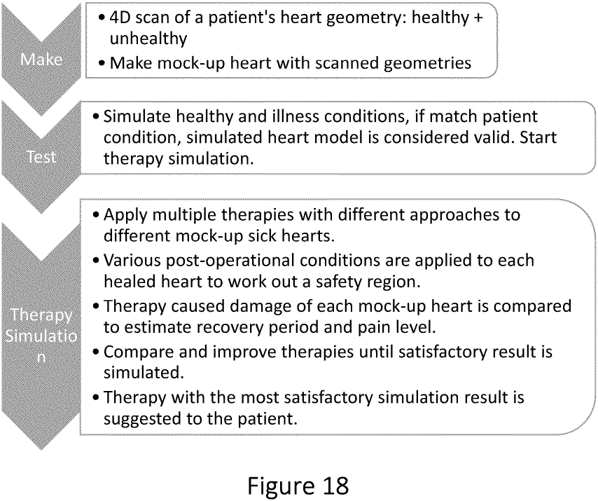

FIG. 18 shows the simulation procedure of prognosis using the invention. When the therapy outcome of a scheduled treatment is to be predicted, the following steps are taken:

Clinical scan (typically MRI) data of the patient’s target organ (LV is taken as an example) is obtained from the patient’s historical medical record. The LV geometries scanned including healthy condition and symptomatic condition are used to 3D print the moulds of mock-up heart components, such as LV mould, MV mould.

Mock-up heart components are made, by way of example, with silicone casting into/onto the 3D printed moulds. The silicone is selected to have similar mechanical properties to human tissue. Additional material is to be embedded into the silicone layers to realise calcification, damaged tissue, healthy/broken chordae, where necessary. Healthy mock-up heart chambers and unhealthy mock-up heart chambers are made respectively.

Run separate simulations under healthy and unhealthy conditions. In healthy condition simulation, the mock-up chambers with healthy geometry are installed with the healthy settings which are heart function parameters obtained from medical records during healthy times, such as cardiac output, heart rate, heart chamber beat waveform, volume pumped per beat, blood pressure. The output of the simulation is illness severity indicators such as effective orifice area, mitral regurgitation fraction, which are to be compared with the healthy medical records. It is expected that the simulation output matches the healthy medical records, otherwise adjustments are to be made such as compliance chamber and blood viscosity until a match is observed. The healthy condition simulation acts as a benchmark against which the results of trial therapies can be compared. The healthy condition simulation also allows a determination to be made as to whether the simulation procedure is likely to be effective. If it is not possible to adjust the parameters of the healthy condition simulation so that the simulation condition output matches the healthy medical records, subsequent simulation will not be sufficiently realistic to determine whether trial therapies are effective. Simulation for unhealthy condition has the same procedure, except that the medical records to be used as settings and expected outcome are from unhealthy data.

While unhealthy condition is applied to the simulator, multiple therapies are to be applied to the mock-up heart chambers, aiming to improve the simulation output (symptom severity) without adjusting the settings. Therapy could be open surgeries such as surgical MV repair and replacement, or minimally invasive therapies such as transcatheter valve repair and replacement. For minimally invasive therapies that do not require the heart to stop beating during the therapy, mock-up heat chambers must keep beating during the therapy as well.

For each therapy, multiple trials with different approaches can be done to different mock-up chambers. For example, it has been reported by researchers that the angle, size and geometry of heart implantations can cause different results.

Once a therapy simulation is completed, various settings are to be applied, including at rest condition and exercise condition, and any possible condition that the patient could face during the rest of his/her life, to provide the safety region for post-operational life.

Once all possible conditions are simulated, the mock-up chamber is to be taken out for future comparison. It will be obvious to see among different mock-up chambers following each therapy, which has suffered the most damage, pain and longest recovery period. Such information is part of the simulation results.

Simulated therapies’ and their results are to be compared and discussed. Innovative improvement can be simulated in more rounds of simulation until a satisfactory result with acceptable risk is achieved.

The procedure above can be applied for the R&D of innovative therapy such as novel implantations.

### Other

The foregoing description has been given by way of example only and the scope of the invention is not limited to the described embodiments. The skilled addressee will appreciate that modifications can be made within the scope of the invention as defined by the appended claims.

For example, it is possible to provide a simulator with a single chamber container and a single mock-up chamber. The mock-up chamber may have a single inlet/outlet, or none, and need not be provided with a valve. Where two mock-up chambers are provided, it is not necessary for both of them to be provided with brachia 13A.

In one aspect, the invention involves providing arms for controlling movement of the positions of points on the mock-up chamber during simulation. Where a valve is provided, it is not necessary to be able to control the position of a mock-up papillary muscle during simulation, or indeed at all.

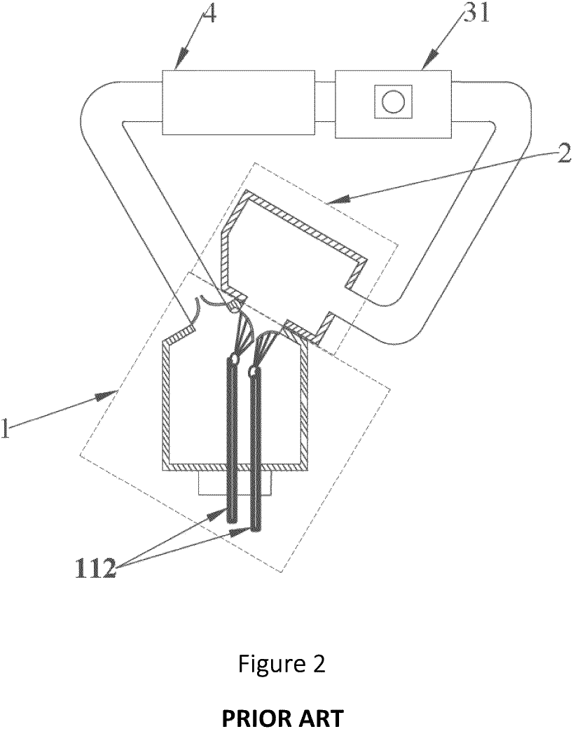

Similarly, other aspects of the invention involve controlling the position of a mock-up tissue, in a specific embodiment a PM tip, with a holder that can be moved (either before or during simulation). Preferably, the PM holder comprises a jointed arm for movement in three dimensions with 6 degrees of freedom (each of the brachium 13B and the forearm 14 can move with 3 degrees of freedom). The mock-up tissue can be moved independently of the chamber container in which it is provided, and even independently of the chamber within which the mock-up tissue (e.g. PM 151) is situated, if there is one. Thus, it should be clear that the provision of a mock-up chamber in addition to the chamber container is not essential to this aspect of the invention. As such, the brachium 13A, 13C or the holder 111 of the present invention could be used to control the position of the papillary muscle tips instead of the papillary muscles controlling arms 112 in FIG. 2, which have a fixed position during simulation and can only be moved up or down.

The invention is not limited to simulation of the left side of the heart, and can also be used to simulate the right side of the heart. It can also be used to simulate the whole of the heart. In any such simulator, any one or more of the mock-up chambers and valves can be controlled with a brachium 13A, 13C or a holder 111, whether for holding a mock-up papillary muscle or another component.

Other organs, such as a lung or a pair of lungs, a kidney or a pair of kidneys, and the bladder, or combinations of organs may also be simulated with the present invention. As such it will be apparent that it may not be necessary to include a valve or even an inlet or outlet in a simulator according to the present invention. It may also be possible to include a single orifice that acts as both the inlet or outlet.

### PBS With Alternative Parts

1 Left Ventricle (LV) Simulation Block
11 LV chamber container
12 mock-up LV chamber
13 brachium could be either 13A (standing alone), 13B (compatible with 14) or 13C (with ball joint)
   131 rotational joint
   132 tube
   133 rod could be 133A without 138, or 133B with 138, or 133C without 138 but compatible with ball joint on 134
   134 clamping twin plates assembly could be a plate pasted to 133A without any joint 134, or with a part joint 134
      1341 upper plate
      1342 lower plate
   135 joint controlling wires
   136 extension controlling wire
   137 spring
   138 holes (only in 1.3B and 14)
   139 ball joint (only for 13C)
14 forearm
   141 rotational joint
   142 tube
   143 rod
   144 connection pad
      1441 threaded holes
   145 joint controlling wires
   146 extension controlling wire
   147 spring
   148 screw
15 mock-up MV
   151 PM
   152 chordae
   153 leaflets
   154 MA
16 AV
17 aorta
18 media liquid
19 mock-up blood
110 motor set
111 PM holder
112 PM controlling arm with fixed position

2 LA Simulation Block
21 LA chamber container
22 mock-up LA
23 mock-up Pulmonary vein

3 pumps

31 mock-up blood pump

4 compliance assembly

5 data system

1. A simulator comprising:
a chamber container;
a flexible simulation chamber disposed inside the chamber container and simulating a chamber of a living body;
at least one chamber arm disposed inside the chamber container, a distal end of the chamber arm being fixed relative to a point on the simulation chamber and a position of the distal end being controllable from a proximal end; and
control means for controlling a simulation procedure to simulate movement of the chamber of the living body, the control means being adapted to change the position of the distal end during the simulation procedure.

2. A simulator according to claim 1, wherein:
the proximal end of the chamber arm is fixed to a wall of the chamber container;
a respective control motor is disposed adjacent the wall of the chamber container and connected to the proximal end; and
the control means is arranged to control the control motor to change the position of the distal end of the chamber arm.

3. A simulator according to claim 1, wherein the chamber arm comprises:
a chamber arm rotational joint towards the proximal end;
chamber arm fixing means towards the distal end for fixing to the simulation chamber; and
a chamber arm extendable rod between the chamber arm rotational joint and the chamber arm fixing means.

4. A simulator according to claim 3, wherein the chamber arm fixing means comprises plates arranged on opposite sides of the simulation chamber for clamping the simulation chamber.

5. A simulator according to claim 3, wherein the chamber arm fixing means is attached to the extendable rod with a ball joint.

6. A simulator according to claim 3, wherein the position of the or each rotational joint is controlled by respective j oint controlling wires and the extension of the or each extendable rod is controlled by a respective extension controlling wire.

7. A simulator according claim 1, wherein
the simulation chamber simulates the left ventricle; and
the control means is arranged to control the position of the distal end of the chamber arm to cause the left ventricle to rotate about a basal-apical axis in synchronisation with a periodical beating movement of the ventricle during the simulation procedure.

8. A simulator according to claim 1 for simulating at least one of the left heart and the right heart.

9. A simulator comprising:
a flexible simulation chamber simulating a chamber of a living body, the simulation chamber having at least one opening;
a valve comprising leaflets;
a simulation papillary muscle; and
chordae connecting the simulation papillary muscle and the leaflets;
at least one valve arm extending through the simulation chamber, so that the distal end of the valve arm is disposed in the simulation chamber and proximal end is disposed outside the simulation chamber, a distal end of the valve arm being fixed relative to the simulation papillary muscle and a position of the distal end of the valve arm being controllable from a proximal end; and
control means for controlling a simulation procedure.

10. A simulator according to claim 9, wherein the position of the distal end is adjustable to simulate end-systole positions of the papillary muscle of the living body.

11. A simulator according to claim 9, the control means being adapted to change the position of the distal end of the valve arm during the simulation procedure.

12. A simulator according to claim 9, wherein the position of the distal end of the valve arm can be controlled without affecting movement of the simulation chamber.

13. A simulator according to claim 12, wherein the valve arm comprises:
a first rotational joint disposed inside the simulation chamber;
first fixing means towards the distal end for fixing to the simulation papillary muscle; and
a first extendable rod between the first rotational joint and the fixing means.

14. A simulator according to claim 13, wherein the valve arm further comprises:
a second rotational joint towards the proximal end and disposed outside the simulation chamber;
second fixing means between the first and second rotational joints for fixing to the simulation chamber; and
a second extendable rod between the second rotational joint and the second fixing means.

15. A simulator according to claim 9, further comprising:
a second simulation chamber, wherein the first and second simulation chambers are connected via the opening with the valve in between them.

16. A simulator according to any one of claim 9, further comprising:
a chamber container in which the simulation chamber is disposed.

17. A simulator according to claim 16, wherein
the proximal end of the valve arm is fixed to a wall of the chamber container;
a control motor is disposed adjacent the wall of the chamber container and connected to the proximal end; and
the control means is arranged to control the control motor to change the position of the distal end of the valve arm.

18. A simulator according to claim 9, further comprising:
at least one chamber arm disposed inside the chamber container, a distal end of the chamber arm being fixed relative to a point on the simulation chamber and a position of the distal end being controllable from a proximal end,
the control means being adapted to change the position of the distal end of the chamber arm during the simulation procedure.

19. A chamber arm for a simulator having a simulation chamber for simulating a chamber of a living body, the arm comprising:
first fixing means towards the distal end for fixing to a portion of the simulation chamber;
a first rotational joint towards the proximal end; and
a first extendable rod between the first rotational joint and the first fixing means,
wherein a position of the distal end is controllable from a proximal end.

20. A chamber arm according to claim 19, further comprising wall fixing means for fixing the proximal end of the valve arm to a wall of a chamber container containing the simulation chamber.

21. A chamber arm according to claim 19, wherein the position of the first rotational joint is controlled by joint controlling wires and the extension of the first extendable rod is controlled by an extension controlling wire.

22. A chamber arm according to claim 19, further comprising a control motor at the proximal end, the control motor being controllable to change the position of the distal end of the arm.

23. A chamber arm according to claim 19, wherein the fixing means comprises plates arranged on opposite sides of the simulation chamber for clamping the simulation chamber.

24. A chamber arm according to claim 19, wherein the first fixing means is attached to the first extendable rod with a ball joint.

25. A valve arm comprising:
a chamber arm according to claim 9;
a second extendable rod between the first fixing means and the distal end of the valve arm;
second fixing means towards the distal end of the valve arm for fixing to a simulated papillary muscle in the simulation chamber; and
a second rotational joint between the second extendable rod and the first fixing means.

26. A valve arm according to claim 25, wherein the position of the second rotational joint is controlled by respective j oint controlling wires and the extension of the second extendable rod is controlled by a respective extension controlling wire.
